(19) United States
(12) Patent Application Publication
(10) Pub. No.: US 2011/0276083 A1
(43) Pub. Date: Nov. 10, 2011

(54) BENDABLE SHAFT FOR HANDLE POSITIONING

(75) Inventors: Frederick E. Shelton, IV, Hillsboro, OH (US); Christopher W. Widenhouse, Clarksville, OH (US); Jerome R. Morgan, Cincinnati, OH (US)

(73) Assignee: ETHICON ENDO-SURGERY, INC., Cincinnati, OH (US)

(21) Appl. No.: 12/775,699

(22) Filed: May 7, 2010

(51) Int. Cl. A61B 17/00

(52) U.S. Cl. 606/205

(57) ABSTRACT

Devices and related methods are disclosed that generally involve elongate surgical instruments that include at least one bendable region to allow the instrument to be bent for improved triangulation, visualization, comfort, and/or maneuverability. In one aspect, a surgical device is provided that includes an elongate body having a proximal end and a distal end, a handle coupled to the proximal end of the elongate body, and an end effector having movable jaws, the end effector being coupled to the distal end of the elongate body. The elongate body includes at least one non-resilient bendable region.

### FIELD

The present invention relates to methods and devices for performing surgical procedures, and in particular to methods and devices for minimally-invasive surgery.

### BACKGROUND

Many surgical procedures involve inserting various instruments through the working channel of a surgical access device. The instruments are used to view, engage, and/or treat tissue within a body cavity or other surgical site to achieve a diagnostic or therapeutic effect. In laparoscopic abdominal procedures for example, the abdominal cavity is generally insufflated with CO₂ gas to a pressure of around 15 mm Hg. The abdominal wall is pierced and a plurality of tubular cannulas, each defining a working channel, are inserted at various points into the abdominal cavity. A laparoscopic telescope connected to an operating room monitor can be used to visualize the operative field and can be placed through one of the cannulas. Other laparoscopic instruments such as graspers, dissectors, scissors, retractors, etc. can be placed through the other cannula(s) to facilitate various manipulations by the surgeon. In this type of procedure, because of the positioning of the cannulas, it can be relatively easy to “triangulate” the tips of two separate instruments (e.g., bring the tips together at a single point within the abdominal cavity). For example, a first instrument could be passed through a cannula in the left side of the patient's abdomen and operated with the surgeon's left hand while a second instrument could be passed through another cannula in the right side of the patient's abdomen and operated with the surgeon's right hand. The surgeon can then easily bring the tips of the two instruments together at an internal point, e.g. in the center of the patient's abdomen. A laparoscope viewing instrument can also be passed through a third cannula, positioned for example in the center of the patient's abdomen, such that the tips of the two instruments can be easily visualized from above.

In other surgical procedures, however, visualization and triangulation is not as straightforward. For example, in Single Incision Laparoscopic Surgery (SILS) or Single Site Laparoscopic Surgery (SSLS), a single laparoscopic entry point is formed (e.g., through the navel). An access device having one or more working channels, and typically a plurality of working channels, is then installed in the entry point and all instruments required for performing the surgery are inserted through this same access device. In such procedures, the elongate shafts of the various instruments end up being generally parallel to one another while inserted through the access device. This can make it very difficult to triangulate the tips of two instruments within the abdominal cavity, especially if the instruments do not have distal articulation capabilities. In addition, since the viewing scope is inserted generally along the same axis as the various other instruments, it can be difficult or impossible to see the tips of the instruments. Furthermore, the handles of the various instruments often end up being positioned in close proximity to one another. Interference between the handles and/or the positioning of the handles can limit maneuverability and/or lead to discomfort for the surgeon. These problems can unduly lengthen the duration of the surgery, potentially increasing the risk of patient complications. Also, in cases where it is impossible to achieve adequate triangulation and/or visualization, a second or even third entry point must be formed, increasing trauma to the patient and creating additional scars.

Even in multiple-incision procedures or where triangulation and visualization is possible (for example where one or more of the devices includes a distal articulation capability), triangulation, visualization, comfort, and maneuverability can still be sub-optimal.

Accordingly, methods and devices are needed for enhancing the ability to triangulate and visualize surgical instruments and to improve surgeon comfort and instrument maneuverability. Furthermore, since each surgery and each patient is slightly different, there is a need for devices that can be customized at the time of surgery.

### SUMMARY

The devices and methods disclosed herein generally involve elongate surgical instruments that include at least one bendable region to allow the instrument to be bent for improved triangulation, visualization, comfort, and/or maneuverability.

In one aspect, a surgical device is provided that includes an elongate body having a proximal end and a distal end. The device can include a handle coupled to the proximal end of the elongate body and an end effector coupled to the distal end of the elongate body. In one embodiment, the end effector can have movable jaws. The elongate body can include at least one non-resilient bendable region.

In one embodiment, the end effector is coupled to the distal end of the elongate body at an articulation joint. The at least one bendable region can be located proximal of the articulation joint. The at least one bendable region can include a first bendable region positioned adjacent to the proximal end of the elongate body and/or a second bendable region positioned adjacent to the distal end of the elongate body.

In one embodiment, the handle can be positioned at an angle of up to 45 degrees in any direction with respect to the longitudinal axis of the body by bending the at least one bendable region.

In another embodiment, the device can also include an actuation member extending through the elongate body and configured to open and close the jaws. The actuation member can be, for example, a wire and/or a bar having a rectangular cross section. In one embodiment, the actuation member includes a first rectangular bar, a wire, and a second rectangular bar, the first rectangular bar being coupled to the end effector and to a first end of the wire, and the second rectangular bar being coupled to the handle and to a second, opposite end of the wire, the wire being disposed within the bendable region of the elongate body. The actuation member can include a relief to permit translation of the actuation member with respect to the elongate body while the elongate body is bent at the bendable region. The device can also include a spring disposed between the actuation member and a trigger element mounted on the handle, the trigger element being configured to longitudinally translate the actuation member with respect to the elongate body.

In one embodiment, the elongate body can include a first section that is formed of a substantially rigid material and a second section that is formed of a deformable material, the second section forming the at least one bendable region. The deformable material can be, for example, a fully annealed aluminum.

In another embodiment, the bendable region can be in the form of a reduced cross-sectional diameter formed along a length of the elongate body.

In another embodiment, the bendable region can be a single, unitary, integral, and/or one-piece structure.

In another aspect, a surgical instrument is provided that includes an elongate body having a proximal end, a distal end, and a longitudinal axis. The instrument also includes a handle coupled to the proximal end of the elongate body and an actuation element disposed within the elongate body, the actuation element being configured to translate substantially along the longitudinal axis of the elongate body. The instrument also includes at least one bendable region configured to allow the handle to be positioned at a non-zero angle with respect to the elongate body while the distal end of the elongate body is inserted in a body cavity.

In yet another aspect, a method for manipulating instruments is provided that includes inserting first and second elongate instruments through at least one working channel of an access device to position distal ends of the first and second instruments within a body cavity and bending at least one of the first and second instruments at a non-resilient bendable region located outside of the body cavity to move a proximal end of the first instrument away from a proximal end of the second instrument. In one embodiment, the at least one instrument is bent prior to insertion through the at least one working channel. In another embodiment, the method can include using a tool to bend the at least one instrument in a controlled manner, the tool being keyed to a shape of the bendable region of the instrument. The method can also include longitudinally translating an actuation element disposed within the at least one instrument while the at least one instrument is bent. In another embodiment, the at least one instrument can include a plurality of non-resilient bendable regions and the method can include forming multiple bends in the at least one instrument.

In a still further aspect, a method for manipulating an instrument is provided that includes inserting an elongate instrument through a working channel of an access device to position a distal end of the instrument within a body cavity. The method can also include engaging a portion of the instrument disposed within the body cavity with a bending tool to bend the instrument at a non-resilient bendable region. In one embodiment, the method can also include articulating the distal end of the instrument about an articulation joint after bending the instrument at the bendable region.

### BRIEF DESCRIPTION OF THE DRAWINGS

The invention will be more fully understood from the following detailed description taken in conjunction with the accompanying drawings, in which:

FIG. 1A is a perspective view of a surgical device according to one embodiment of the present invention;

FIG. 1B is a cross-sectional side view of the surgical device of FIG. 1A;

FIG. 2A is a perspective view of a surgical device according to another embodiment of the present invention;

FIG. 2B is a side view of the surgical device of FIG. 2A;

FIG. 3A is a side view of a surgical device having a plurality of bendable regions according to another embodiment of the present invention;

FIG. 3B is a side view of a surgical device having a plurality of bendable regions according to another embodiment of the present invention;

FIG. 3C is a side view of a surgical device having a plurality of bendable regions according to another embodiment of the present invention;

FIG. 3D is a side view of a surgical device having a plurality of bendable regions according to another embodiment of the present invention;

FIG. 3E is a side view of a surgical device having a plurality of bendable regions according to another embodiment of the present invention;

FIG. 4 is a side view of a surgical device according to another embodiment of the present invention;

FIG. 5 is a side view of a surgical device according to another embodiment of the present invention;

FIG. 6A is a cross-sectional side view of one embodiment of an actuation member disposed within a surgical device and including a relief formed therein;

FIG. 6B is a cross-sectional side view of the surgical device and actuation member of FIG. 6A shown in a bent configuration;

FIG. 7A is a cross-sectional side view of another embodiment of an actuation member disposed within a surgical device and including first and second rectangular bars joined by a length of wire;

FIG. 7B is a cross-sectional side view of the surgical device and actuation member of FIG. 7A shown in a bent configuration;

FIG. 8A is a side view of a tool for bending a surgical device according to one embodiment of the present invention;

FIG. 8B is another side view of the tool of FIG. 8A;

FIG. 9A is a side view of a surgical device before being bent according to one embodiment of the present invention;

FIG. 9B is a side view of the surgical device of FIG. 9A shown in a bent configuration;

FIG. 9C is a partial cross-sectional side view of the surgical device of FIGS. 9A-9B inserted through a working channel of a surgical access device after being bent;

FIG. 9D is a partial cross-sectional side view of the surgical device and surgical access device of FIG. 9C showing a second surgical device inserted through a second working channel of the surgical access device; and

FIG. 10 is a partial cross-sectional side view of two surgical devices shown partially inserted into a body cavity.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In general, surgical methods and devices are provided that involve elongate surgical instruments that include at least one bendable region to allow the instruments to be bent for improved triangulation, visualization, comfort, and/or maneuverability. A person skilled in the art will appreciate that, while methods and devices are described herein in connection with minimally invasive laparoscopic procedures in the abdominal cavity, the methods and devices can be used in almost any part of a human or animal body and in various other types of surgical procedures. By way of non-limiting example, the devices and methods disclosed herein can be used in the thoracic cavity, pelvic cavity, cranial cavity and/or any of the body's natural orifices and can be used in endoscopic procedures and/or in open surgical procedures.

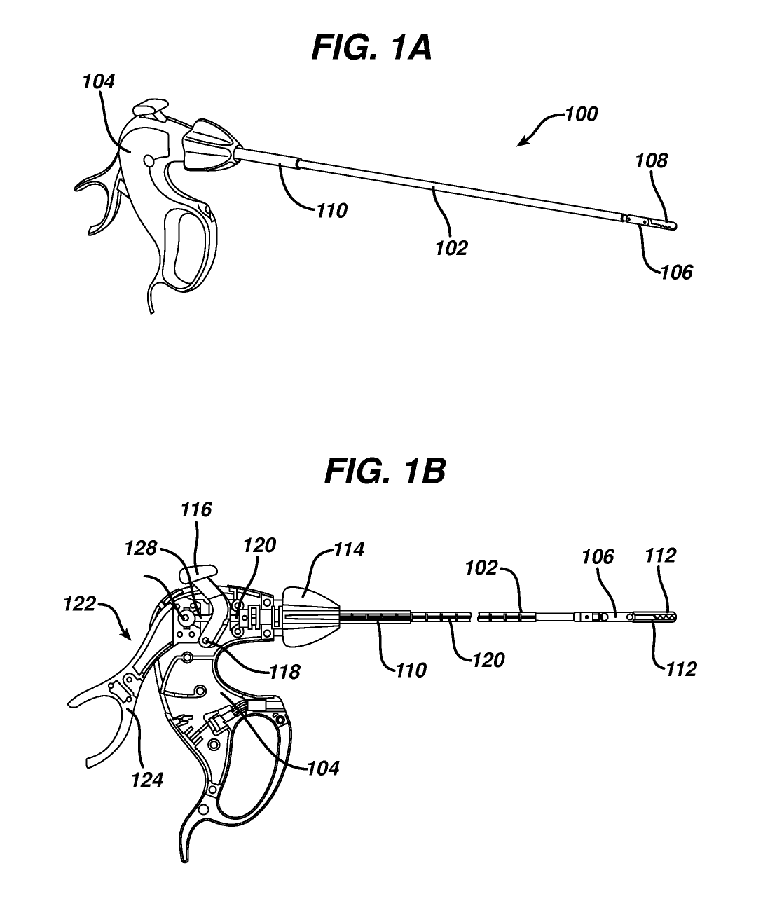

FIGS. 1A and 1B illustrate one exemplary embodiment of a surgical device 100. As shown, the device 100 includes an elongate body 102, a handle 104 coupled to the proximal end of the body 102, and an end effector 108 coupled to the distal end of the body 102 at an articulation joint 106. The device also includes at least one bendable region 110.

The end effector 108 can be virtually any device, structure, or element that is useful in performing surgery. In an exemplary embodiment, the end effector has opposed jaws. For example, the end effector 108 can include one or more graspers, dissectors, Babcocks, and/or scissors. In the illustrated embodiment, a grasper-type end effector 108 is shown having movable jaws 112 that can be selectively opened and closed by a surgeon, as explained in more detail below.

The end effector 108 can be coupled directly to the body 102 or an articulation joint 106 can be provided therebetween. The articulation joint 106 can facilitate angular positioning of the end effector 108 with respect to the body 102. Articulation joints are well known in the art of minimally invasive surgery and any suitable joint can be employed. In the illustrated embodiment, the articulation joint 106 is in the form of a multi-segment pivoting linkage. While not shown, the end effector can also or alternatively be coupled to the distal end of the elongate body by a pivot/rotation joint which allows the end effector to rotate relative to the body 102. A person having ordinary skill in the art will appreciate that the device can include any number of articulation and/or rotation joints at various locations. For example, the proximal end of the body 102 can be rotatably coupled to the handle.

The shaft or body 102 of the device 100 can be a solid structure or it can have one or more lumens or cavities formed therein. In the illustrated embodiment, the body 102 is generally in the form of an elongate rigid tube having a circular cross-section, though various other configurations are also possible. The body 102 can be formed from any material or materials that exhibit sufficient rigidity and suitability for surgical applications. For example, the body 102 can be formed from surgical-grade stainless steel. The body 102 can be sized to permit insertion of at least a portion thereof through a working channel of a surgical access device (not shown). The length of the body 102 can be chosen based on a variety of factors, including the specific anatomy of the patient, the location of the surgical site, the preferences of the surgeon, the type of procedure being performed, etc. In one embodiment, the body 102 can have an outside diameter of approximately 5 mm, an inside diameter of approximately 4.2 mm, and a length of approximately 33 cm.

A handle 104 can be directly or indirectly coupled to the proximal end of the body 102. The handle 104 can include various components for actuating, articulating, rotating, and/or otherwise manipulating the end effector 108. For example, as shown, a rotatable nozzle 114 can be mounted to the distal end of the handle 104. The nozzle 114 can be operably coupled to the end effector 108 (e.g., via the body 102 or one or more components disposed therein) such that rotation of the nozzle 114 with respect to the handle 104 results in a commensurate rotation of the end effector 108 with respect to the handle 104. In one embodiment, the nozzle 114 can be rotated 360 degrees with respect to the handle 104.

The handle 104 can also include a pivot arm 116 configured to articulate the end effector 108 with respect to the elongate body 102 via the articulation joint 106. In the illustrated embodiment, rotation of the pivot arm 116 with respect to a first pivot pin 118 causes longitudinal translation of an articulation member 120 coupled thereto and disposed within the elongate body 102. The articulation member 120 is coupled at its distal end to the articulation joint 106 such that longitudinal translation of the articulation member 120 with respect to the elongate body 102 causes the articulation joint 106 (end the end effector 108 coupled thereto) to pivot or otherwise articulate. In the illustrated embodiment, the articulation member 120 is in the form of an elongate bar having a rectangular cross-section. As will be explained below, the articulation member 120 can have a variety of other forms as well, including one or more wires, bars, or combinations of such structures. In one embodiment, the articulation member 120 is in the form of a rectangular bar having a 3:1 ratio of height to width.

The handle 104 can also include a trigger element 122 to actuate the end effector 108. For example, in the illustrated embodiment, a thumb lever 124 can be operable to open and close the jaws of the graspers. The thumb lever 124 can be mounted to the handle 104 with a second pivot pin 126 and it can be coupled to the proximal end of an actuation member 128 extending through the elongate body 102, substantially parallel to the articulation member 120. The actuation member 128 can in turn be coupled at its distal end to the end effector 108. In use, movement of the thumb lever 124 causes the actuation member 128 to be translated longitudinally within the elongate body 102 to actuate the end effector 108. The actuation member 128 can be in the form of a wire, a bar, or some combination thereof. The actuation member 128 can run alongside the articulation member 120 as shown or it can be disposed therein or therearound (e.g., positioned coaxially with the articulation member 120).

The device 100 can also include at least one bendable region 110. The bendable region or regions 110 can be located anywhere along the length device 100, including without limitation at various points along the length of the body 102, between the body 102 and the articulation joint 106, and between the body 102 and the handle 104. The bendable region or regions 110 can be formed integrally with the body 102 and/or can be separate components coupled thereto. In embodiments in which the at least one bendable region 110 is a separate component or components, any of a variety of techniques known in the art can be used to couple the at least one bendable region 110 to the body 102, such as welding or bonding using a suitable adhesive. The bendable region 110 and the body 102 can also engage one another via a threaded interface, friction fit, or other joining element known in the art. The body 102 can optionally be formed of several discrete pieces separated and joined together by one or more bendable regions 110.

The bendable region 110 can be either resilient or non-resilient. Thus, the bendable region 110 can be configured to bend and maintain a bent position after bending forces are removed. The bendable region 110 can be configured to bend multiple times at multiple angles before failing.

In one embodiment, the elongate body 102 is formed of a substantially rigid material while the bendable region 110 is formed of a deformable (e.g., plastically-deformable) and/or flexible material. For example, the elongate body 102 can be formed of surgical-grade stainless steel and the bendable region 110 can be formed of fully annealed aluminum. While aluminum is preferred for the bendable region 110, a variety of other materials can also be used. For example, various hard rubbers, aluminum alloys, or other materials having sufficient rigidity to support the weight of organs or other materials that may be grasped or manipulated with the device can be used. In such embodiments, the wall thickness of the bendable region 110 can be slightly thicker than that of the elongate body 102 if necessary to provide added strength to the bendable region 110. For example, since materials such as aluminum generally have less compressive loading ability than steel, the aluminum section can be formed to have a greater thickness. Thus, the inside diameter of the bendable region 110 can be less than that of the elongate body 102. Alternatively, the inside diameter of the bendable region 110 can be the same as the inside diameter of the elongate body 102, and the outside diameter of the bendable region 110 can instead be increased. In one embodiment, the substantially rigid portion of the elongate body 102 has a wall thickness of approximately 0.0015 inches and the deformable/flexible portion of the body 102 (i.e. the bendable region 110) has a wall thickness of approximately 0.0045 inches.

In the embodiment shown in FIGS. 1A and 1B, a single bendable region 110 is provided immediately adjacent and distal to the nozzle 114. The proximal end of the bendable region 110 can be fixedly or rotatably coupled to the nozzle 114 and the distal end of the bendable region 110 can be fixedly or rotatably coupled to the body 102. Since it is rarely necessary to fully insert the body 102 through the surgical access device (e.g., all the way up to the nozzle), this positioning of the bendable region 110 can advantageously permit bending of the device 100 without substantially limiting the depth to which the device 100 can be inserted. The bendable region 110 can be approximately 2 inches in length and can form the proximal-most portion of the body 102. As a result, the bendable region 110 can be bent outside of the patient's body while the end effector and distal end of the body 102 are disposed within the patient's body.

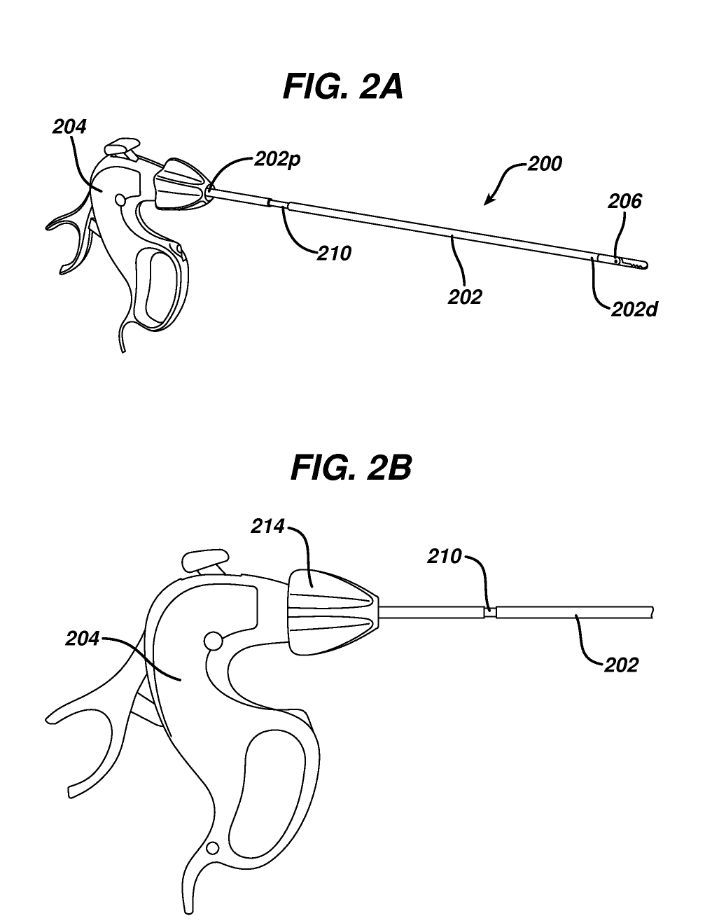

In other embodiments, as shown for example in FIGS. 2A and 2B, a device 200 can include one or more bendable regions 210 in the form of a section of the elongate body 202 having a reduced cross-sectional diameter. When bending forces are applied to the device 200, the tendency will be for the body 202 to bend at the location of reduced cross-sectional diameter 210. The bendable region 210 can be located anywhere along the length of the elongate body 202, including at a terminal end thereof. In one embodiment the bendable region 210 can be positioned at a location closer to the proximal end 202p of the elongate body 202 than the distal end 202d of the elongate body 202. In an exemplary embodiment, the bendable region 210 is approximately 0.5 inches long and is positioned approximately 3 inches from the nozzle 214 and 25-30 centimeters from the articulation joint 206. In one embodiment, the section of reduced cross-section 210 is stepped down with respect to the adjacent portions of the elongate body 202 by a depth approximately equal to the wall thickness of the elongate body 202.

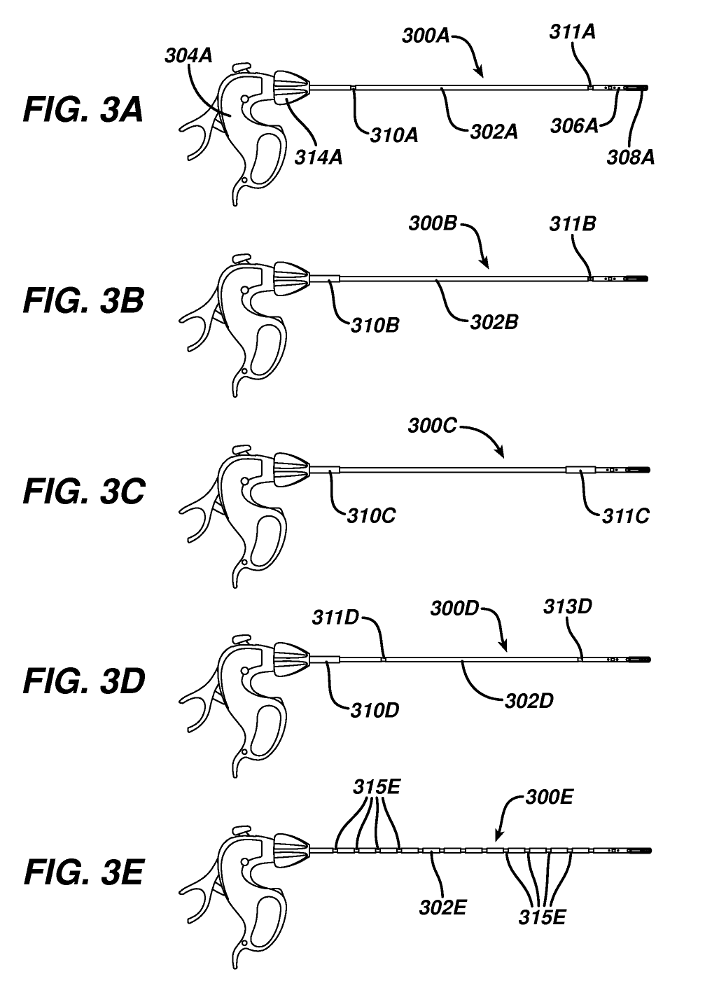

As shown in FIGS. 3A-3E, surgical devices according to the present invention can also include a plurality of bendable regions. In FIG. 3A, the elongate body 302A of a device 300A includes first and second bendable regions 310A, 311A. The first bendable region 310A is positioned near the proximal end of the body 302A (e.g., approximately 3 inches from the end of the nozzle 314A and 25-30 centimeters from the articulation joint 306A), and can permit positioning of the handle 304A at an angle with respect to the body 302A. The second bendable region 311A is positioned adjacent to the distal end of the body 302A (e.g., approximately 1 inch from the articulation joint 306A and 30-35 centimeters from the nozzle 314A). This second bendable region 311A can be bent prior to insertion of the device 300A into a surgical access device, and it can enhance the triangulation capabilities of the device 300A, particularly in embodiments that do not include an articulation joint 306A between the body 302A and the end effector 308A or where the range of articulation provided by the articulation joint 306A is limited.

In FIG. 3B, a device 300B is shown including a first bendable region 310B formed of a flexible and/or deformable material and a second bendable region 311B formed from a length of the body 302B having a reduced cross-section.

FIG. 3C illustrates another exemplary embodiment of a device 300C in which first and second bendable regions 310C, 311C are provided, each bendable region 310C, 311C being formed of a flexible and/or deformable material.

As shown in FIG. 3D, a device 300D can include a first bendable region 310D formed of a flexible and/or deformable material. The device can also include second and third bendable regions 311D, 313D formed from a length of the body 302D having a reduced cross-section.

FIG. 3E illustrates yet another embodiment of a device 300E having several bendable regions 315E equally spaced along the entire length of the body 302E.

It will be appreciated that a variety of other permutations and combinations of bendable regions can be incorporated into the devices of the present invention and that the illustrated embodiments are merely exemplary and are not intended to limit the scope of the present invention.

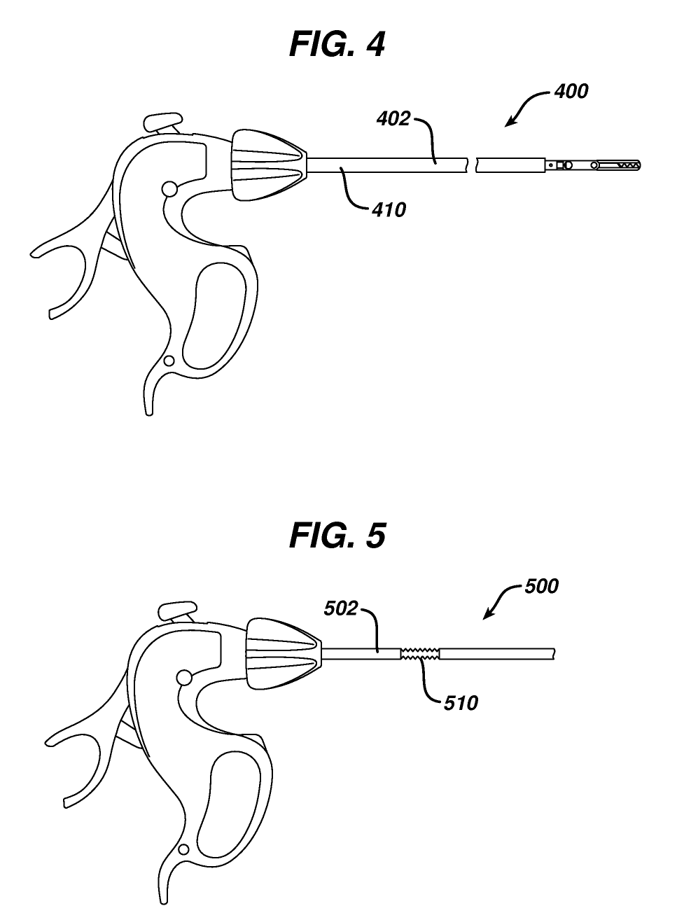

FIG. 4 illustrates another embodiment of a surgical device 400 in which the entire body 402 constitutes a bendable region 410. In the illustrated embodiment, the entire body 402 is formed of a deformable/flexible material, such as fully annealed aluminum. In such embodiments, one or more bends can be formed at any location along the length of the body 402.

FIG. 5 illustrates another embodiment of a surgical device 500 in which the bendable region 510 comprises a section of the elongate body 502 having accordion or bellows-style walls. This section can be formed from a variety of materials, including rubbers, plastics, metals, polyethylene, thermoplastic resins (e.g., Isoplast®), nylon (glass filled or unfilled), and/or combinations thereof. Such a configuration can advantageously permit the device to be bent a virtually unlimited number of times before failure. In an exemplary embodiment, the size and shape of the individual pleats of the bendable region 510 are chosen such that the bendable region 510 is non-resilient and capable of maintaining a bent position, even under the forces typically applied to laparoscopic instruments during surgery.

Providing bendable regions as disclosed herein can advantageously facilitate “on the fly” customization of the device by permitting a surgeon or other user to bend the device as desired before or even during surgery (e.g., while the instrument and/or a distal end thereof is at least partially inserted into a body cavity). Bending the at least one bendable region can permit the handle and/or the end effector to be positioned at a variety of angles with respect to the longitudinal axis of the body. For example, bend angles in the range of about 0 to about 180 degrees, about 0 to about 90 degrees, about 0.1 to about 45 degrees, about 0.1 to about 35 degrees, about 0.1 to about 25 degrees, about 0.1 to about 15 degrees, and/or about 0.1 to about 5 degrees are attainable with the methods and devices disclosed herein.

Furthermore, bending the at least one bendable region can advantageously improve triangulation and visualization of the tip of the device. For example, where a bendable region or regions are provided near the distal end of the elongate body, a surgeon can opt to bend the instrument before insertion into the abdominal cavity. As will be described in more detail below, such a configuration makes it easier to see the distal tip of the device and to bring the distal tip together with the tip or tips of another instrument or instruments.

The instruments and devices disclosed herein can generally be bent at the one or more bendable regions without substantially interfering with the operation of the device (e.g., the ability of the device to articulate, actuate, rotate, or otherwise manipulate the end effector). In one embodiment, the internal components of the device, including for example the actuation member and/or the articulation member, can include various modifications and/or additional features in order to further facilitate bending of the device and operation of the device while bent.

As explained above, the devices disclosed herein can include an actuation member, e.g., in the form of an elongate bar having a rectangular cross-section of a substantially consistent size throughout its length. Since the cross-sectional size of the actuation member is generally much less than the cross-sectional size of the lumen in which it is disposed, the body can typically be bent to an appreciable degree without interfering with the translation of the actuation member. Even when the body is bent enough for there to be an interference between the actuation member and the lumen in which it is disposed, further bending can take place without binding the components. For example, the actuation member can be configured to bend along with the body in which it is disposed in such cases.

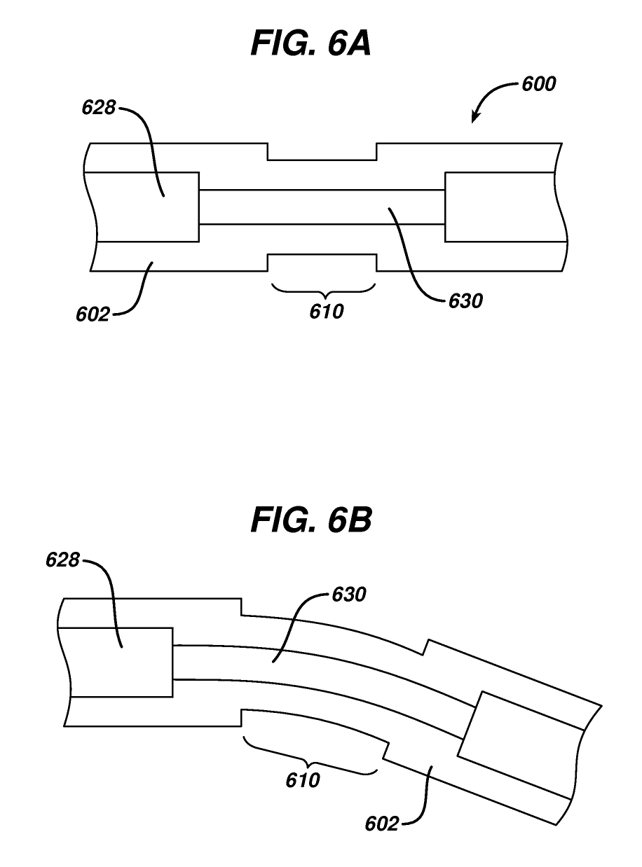

Alternatively, or in addition, in any of the devices disclosed herein, a relief can be formed in the actuation member to provide clearance for further angulation of the body or to reduce or eliminate binding of the actuation member with the surrounding lumen. FIG. 6A illustrates one embodiment of a device 600 in which the actuation member 628 includes an area of reduced cross-section 630. This area 630 can be generally disposed within and/or generally aligned with a bendable region 610 formed in the body 602 of the device 600. In embodiments in which multiple bendable regions 610 are provided, multiple of such reliefs 630 can be formed in the actuation member 628. As shown in FIG. 6B, when the body 602 is bent, the relief 630 in the actuation member 628 can provide additional clearance when translating the actuation member 628 through the bent region 610 of the body 602. The length of the relief 630 can vary, but in an exemplary embodiment it is greater than the length of the bendable region 610 to allow for longitudinal translation of the actuation member 628 with respect to the body 602.

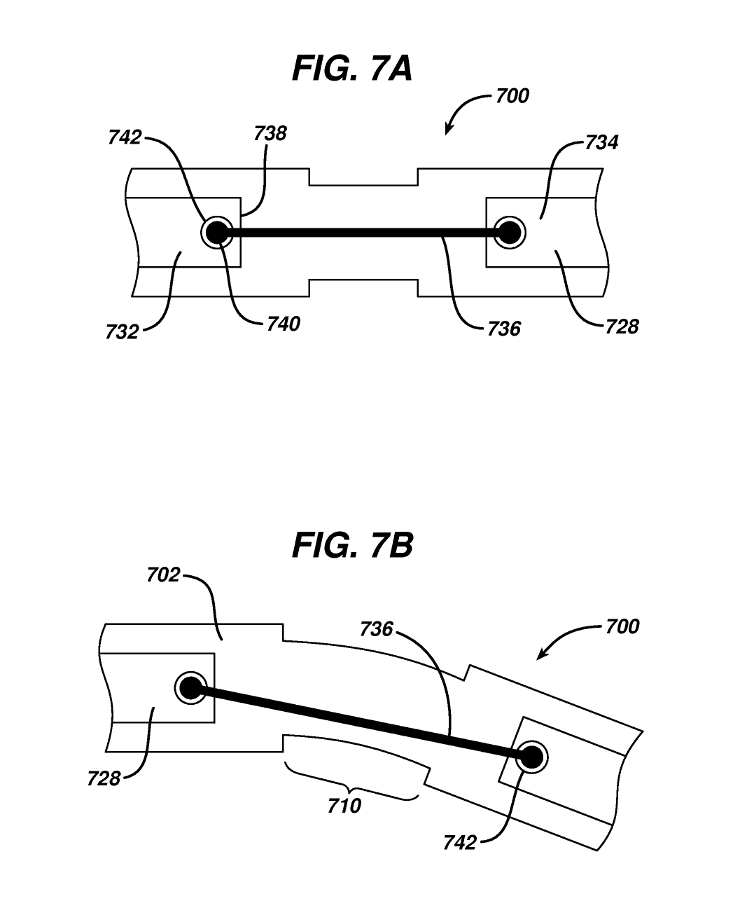

The actuation member(s) of any of the devices disclosed herein can optionally include other features or modifications for improving clearance. For example, the actuation member can include first and second rectangular bars separated by a length of wire. FIG. 7A illustrates one embodiment of a device 700 in which the actuation member 728 comprises a first rectangular bar 732, a second rectangular bar 734, and a length of wire 736. A first end of the wire 736 can be coupled to the distal end 738 of the first rectangular bar 732, for example by forming a ball or stopper 740 on the end of the wire and then threading the wire through a transverse bore hole 742 formed in the distal end 738 of the first rectangular bar 732. A second end of the wire 736 can be similarly coupled to the proximal end of the second rectangular bar 734. Preferably, the gauge of the wire 736 is selected such that the wire 736 has a smaller cross section than that of the first and second rectangular bars 732, 734. In addition, the wire 736 can be flexible. As shown in FIG. 7B when the body 702 is bent at a bendable region 710, the reduced cross-section of the wire 736 provides added clearance for translation of the actuation member 728 through the bent region 710 of the body 702. It will be appreciated that when the body 702 is bent at a greater angle than that pictured, the flexibility of the wire 736 will permit the actuation member 728 to curve or otherwise bend around the bend in the body 702. The length of the wire 736 can vary, but in an exemplary embodiment it is greater than the length of the bendable region 710 to allow for longitudinal translation of the actuation member 728 with respect to the body 702.

The non-illustrated ends of the first and second rectangular bars 732, 734 can be operably coupled to a handle (not shown) and an end-effector (not shown), respectively. Alternatively, other segments of the actuation member can be formed from one or more wires, for example when the device includes a plurality of bendable regions, in which case the first and second rectangular bars could be coupled to one or more of said wires which could in turn be coupled to one or more additional rectangular bars, or to the handle and/or the end effector.

In certain embodiments, one or more springs can be positioned in series between the actuation member and the thumb lever of the trigger element to introduce additional play into the system and to further facilitate translation of the actuation member when the body is bent. In other embodiments, the actuation member can include a constant velocity, universal, or other joint to facilitate continued operation of the device after bending.

It will be appreciated that any of the modifications or features described above with respect to the actuation member can also be applied to the articulation member, as well as any other components that are translated within the interior of the body. The length of such modifications and features can be selected to correspond to the lengths of the bendable regions of the device and in some embodiments can be selected to have a length greater than that of the bendable regions. This latter configuration can provide even further clearance for longitudinal translation of the internal components with respect to the body.

Additionally, any of the internal components of any of the various devices disclosed herein can be formed of and/or coated with a friction-reducing material so as to further facilitate operation thereof while the device is bent at one or more locations.

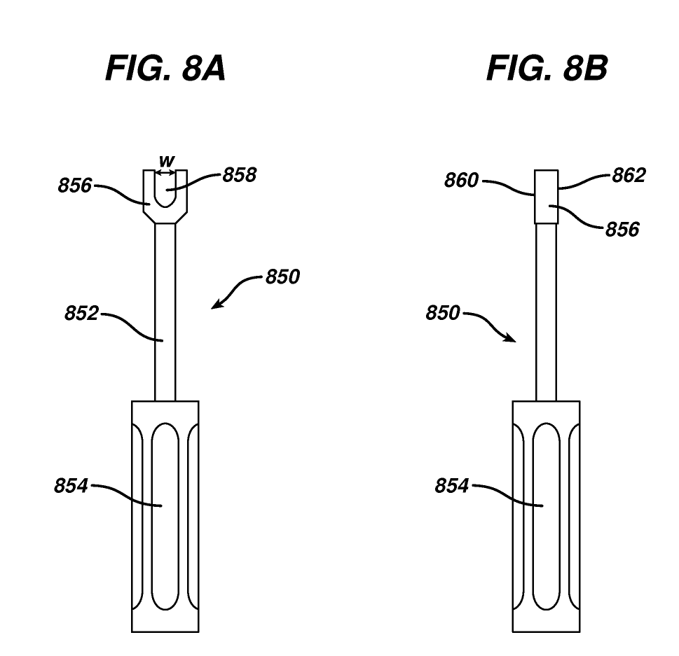

The various devices disclosed herein can also be provided in a kit that further includes at least one tool configured to make bending of the device easier and more controlled. FIGS. 8A and 8B illustrate one embodiment of such a tool 850. As shown, the tool 850 can include a shaft 852 with a handle 854 configured to be grasped by a user. The tool 850 can also include a head 856 for engaging a portion of one or more of the devices disclosed herein. In one embodiment, the head 856 can include a recess 858 having a width W that substantially corresponds to the diameter of a region of reduced cross section formed in the body of a surgical device. Thus, the tool 850 can be keyed to a particular device or to a particular portion of a particular device. As will be described below, the fork-shaped head 856 of the illustrated tool 850 can include bearing surfaces 860, 862 for prying against the raised sections of the elongate body on either side of the necked-down area having a reduced cross-section. In an exemplary embodiment, the bending tool can be sized, shaped, and/or otherwise configured to be inserted through the working channel of an access device, such as a laparoscopic port or an endoscope. In such embodiments, as explained further below, the tool can be used to bend a device that is already inserted through the access device and positioned at least partially within a body cavity.

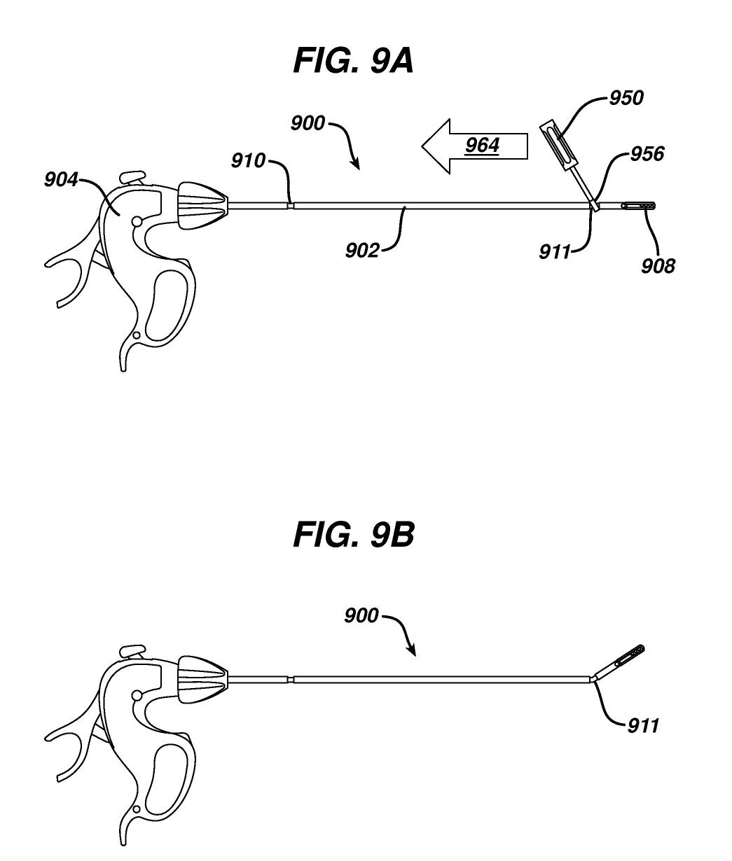

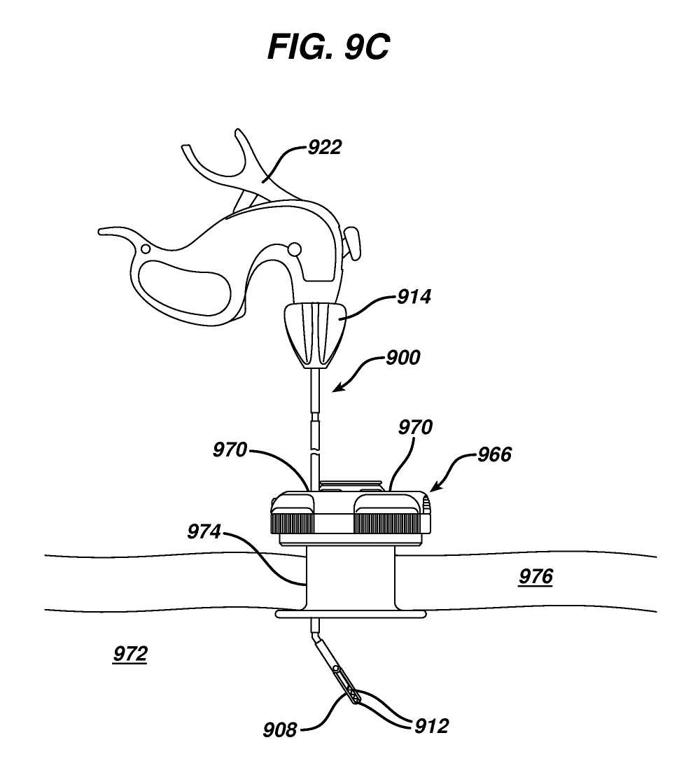

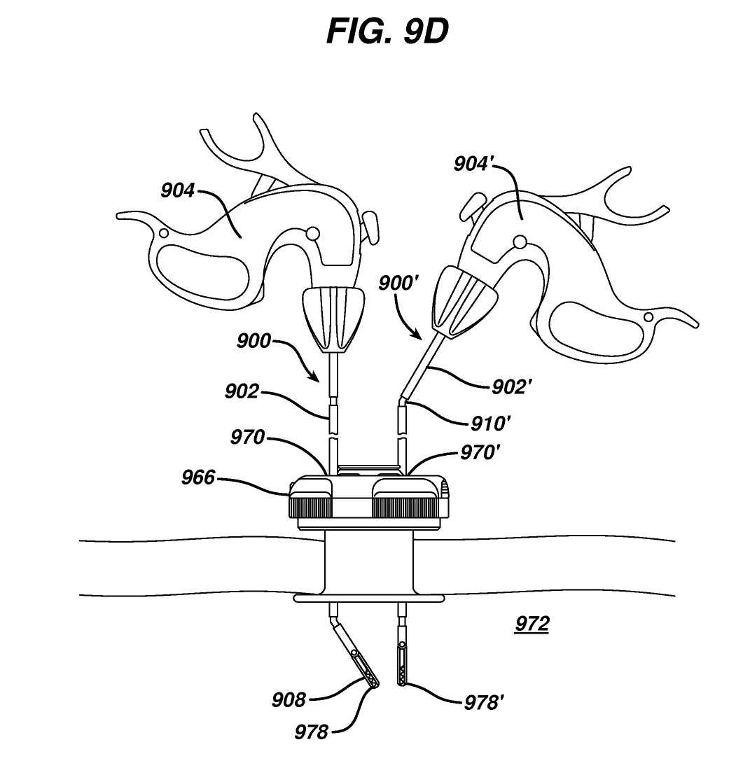

In use, the devices disclosed herein can enable a user perform various surgical tasks with enhanced triangulation, visualization, maneuverability, and operator comfort. FIGS. 9A-9D illustrate one exemplary method of using one or more of the devices disclosed herein.

FIG. 9A illustrates one embodiment of a surgical instrument 900 that includes first and second bendable regions 910, 911 in the form of segments of the body 902 having a reduced cross-section. The instrument also includes an end effector 908 coupled directly to the body 902, without any intermediate articulation joint. As shown, a tool 950 can be used to apply a bending torque to the body 902 of the instrument 900, for example at the location of the second bendable region 911. When a force is applied to the tool 950 in the general direction of the arrow 964, the head 956 of the tool 950 bears against the ridges formed where the body 902 transitions to and from the area 911 having a reduced cross section, thus transferring the force thereto. A user can apply force in this manner until the instrument 900 is bent to the desired degree. It will be appreciated that the instrument 900 can be bent in any direction, 360 degrees around its longitudinal axis. It will also be appreciated that the direction of the bend can be adjusted by adjusting the position of the tool 950.

In the illustrated embodiment, the bendable region 911 is non-resilient, as demonstrated by the fact that the instrument 900 remains bent in FIG. 9B, even after the tool 950 and any forces applied thereby have been removed.

Before or after bending the instrument 900 to the desired degree, it can be inserted through the working channel of a surgical access device and into a body cavity, as shown in FIG. 9C. The surgical access device 966 generally includes a seal housing 968 that defines one or more working channels 970. One or more seals (not shown) can be disposed in the working channel(s) 970 to maintain a seal thereacross to prevent the escape of insufflation gas from a body cavity 972. The seals can be configured to maintain insufflation regardless of whether an instrument is inserted through the working channel(s) 970. The surgical access device 966 can also include a retractor 974 configured to maintain an opening through a tissue wall 976 and/or to help retain the access device 966 in position. The access device 966 can also include various other features that are not shown, such as a valve assembly for supplying or removing insufflation gas from the body cavity 972 and one or more tie-downs for further securing the access device 966 to the tissue wall 976.

As shown, the instrument 900 can be inserted through a first working channel 970 of the surgical access device 966. Once inserted, various surgical manipulations can be performed using the instrument 900, for example by actuating an end effector 908 thereof (e.g., by squeezing a trigger element 922 to selectively open and close the jaws 912 of the end effector 908) and/or by rotating the end effector 908 (e.g., by rotating a nozzle 914).

A second instrument 900′ can also be inserted, either through the same working 970 channel or through a second working channel 970′ in the surgical access device 966, as shown in FIG. 9D. The second device 900′ can be bent or un-bent and can be of a same or different type as the first device 900. In the illustrated embodiment, the second device 900′ is bent at a bendable region 910′ located in the proximal end of the body 902′ (i.e., outside of the patient's body), either before or after insertion of the instrument 900′ through the working channel 970′.

In certain procedures, a viewing scope (not shown) can be inserted through a third working channel (not shown) of the access device 966.

Because the end effector 908 of the first instrument 900 is positioned at an angle with respect to the majority of the body 902 thereof, triangulation and visualization is improved. In other words, even though the instruments 900, 900′ and the viewing scope are inserted through a common incision, it is still possible to see the tips 978, 978′ of the instruments 900, 900′ and to bring the tips 978, 978′ of the two instruments 900, 900′ together to a single point within the body cavity 972.

In addition, because the handle 904′ of the second device is bent with respect to the majority of the body 902′ thereof, maneuverability is improved since interference between the handles 904, 904′ of the two instruments 900, 900′ is avoided. Finally, user comfort can be enhanced, since the bend angles of the instruments 900, 900′ can be customized at any time before or during the surgery.

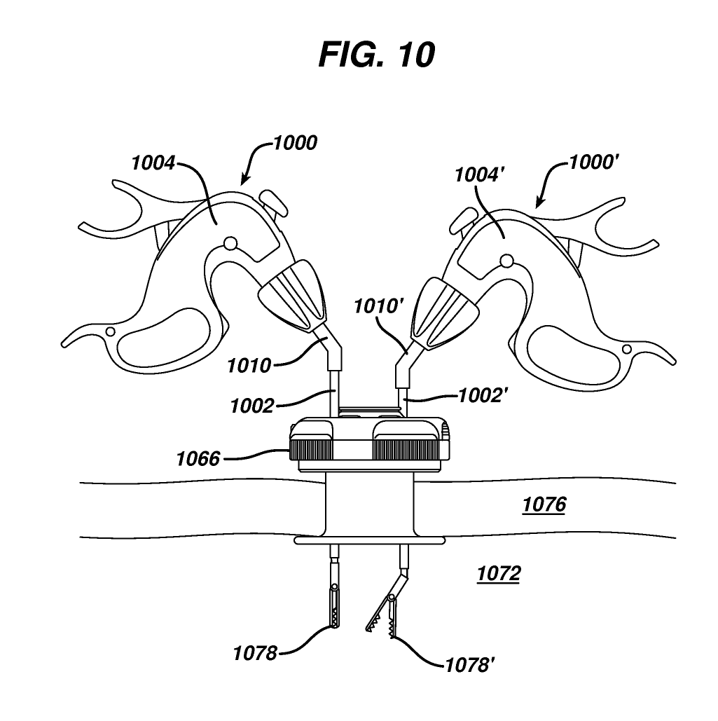

FIG. 10 illustrates another exemplary surgical setup in accordance with the devices and methods disclosed herein. As shown, two bendable instruments 1000, 1000′ can be inserted through a SILS-type access device 1066 to access a body cavity 1072 underlying a tissue wall 1076. The instruments 1000, 1000′ can include sections of deformable and/or flexible material 1010, 1010′ to facilitate bending of the handles 1004, 1004′ with respect to the bodies 1002, 1002′ of the instruments 1000, 1000′. As shown and as explained above, this can advantageously avoid interference between the two handles 1004, 1004′, thereby increasing maneuverability. Furthermore, by positioning the handles 1004, 1004′ off-axis with respect to the instruments 1000, 1000′, triangulation of the surgeon's two hands and the point at which the tips 1078, 1078′ of the instruments 1000, 1000′ converge can be improved.

In another embodiment, at least one instrument disclosed herein can be bent within a body cavity by a bending tool. For example, a bending tool as disclosed herein can be inserted through the same access port or through another port such that its distal end is also positioned within the body cavity. The bending tool can then be used to bend the at least one instrument at a non-resilient bendable region located inside of the body cavity. For example, in the embodiment shown in FIG. 9C, the instrument 900 can optionally be inserted through the access device 966 before being bent. A bending tool (not shown) can then be inserted through a working channel 970 of the access device 966 to bend a portion of the instrument disposed within the body cavity 972. This method can be particularly useful when the diameter of the working channel is not sufficient to bend the instrument before insertion into the body cavity. While the instrument is bent, or before or after the instrument is bent, the instrument can be articulated, actuated, rotated, and/or otherwise manipulated as disclosed herein. To remove the bent instrument, the bending tool can be reinserted through the port (if not already inserted therethrough) and used to bend the instrument back to a substantially un-bent configuration.

A person having ordinary skill in the art will appreciate that various other configurations and manipulations are possible. For example, one or more of the instruments can include a plurality of bendable regions (as shown for example in FIGS. 3A-3E) and thus the methods disclosed herein can involve forming a plurality of bends in the instrument(s), either before inserting the instrument into a surgical site or while the instrument is inserted. Furthermore, it will be appreciated that the ability to actuate, articulate, rotate, and otherwise manipulate the end effectors of the devices disclosed herein still exists after the devices are bent.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the devices disclosed herein are sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical device, comprising:
an elongate body having a proximal end and a distal end;
a handle coupled to the proximal end of the elongate body; and
an end effector having movable jaws, the end effector being coupled to the distal end of the elongate body;
wherein the elongate body includes at least one non-resilient bendable region.

2. The device of claim 1, wherein the end effector is coupled to the distal end of the elongate body at an articulation joint.

3. The device of claim 2, wherein the at least one bendable region is located proximal of the articulation joint.

4. The device of claim 1, wherein the at least one bendable region comprises a first bendable region positioned adjacent to the proximal end of the elongate body.

5. The device of claim 4, wherein the at least one bendable region further comprises a second bendable region positioned adjacent to the distal end of the elongate body.

6. The device of claim 1, wherein the handle can be positioned at an angle of up to 45 degrees in any direction with respect to the longitudinal axis of the body by bending the at least one bendable region.

7. The device of claim 1, further comprising an actuation member extending through the elongate body and configured to open and close the jaws.

8. The device of claim 7, wherein the actuation member is selected from the group consisting of a wire and a bar having a rectangular cross section.

9. The device of claim 7, wherein the actuation member comprises a first rectangular bar, a wire, and a second rectangular bar, the first rectangular bar being coupled to the end effector and to a first end of the wire, and the second rectangular bar being coupled to the handle and to a second, opposite end of the wire, the wire being disposed within the bendable region of the elongate body.

10. The device of claim 7, wherein the actuation member includes a relief to permit translation of the actuation member with respect to the elongate body while the elongate body is bent at the bendable region.

11. The device of claim 7, further comprising a spring disposed between the actuation member and a trigger element mounted on the handle, the trigger element being configured to longitudinally translate the actuation member with respect to the elongate body.

12. The device of claim 1, wherein the elongate body comprises a first section that is formed of a substantially rigid material and a second section that is formed of a deformable material, the second section forming the at least one bendable region.

13. The device of claim 12, wherein the deformable material comprises fully annealed aluminum.

14. The device of claim 1, wherein the bendable region comprises a length of the elongate body having a reduced cross-sectional diameter.

15. A surgical instrument, comprising:
an elongate body having a proximal end, a distal end, and a longitudinal axis;
a handle coupled to the proximal end of the elongate body;
an actuation element disposed within the elongate body, the actuation element being configured to translate substantially along the longitudinal axis of the elongate body; and
at least one bendable region configured to allow the handle to be positioned at a non-zero angle with respect to the elongate body while the distal end of the elongate body is inserted in a body cavity.

16. A method for manipulating instruments, comprising:
inserting first and second elongate instruments through at least one working channel of an access device to position distal ends of the first and second instruments within a body cavity; and
bending at least one of the first and second instruments at a non-resilient bendable region located outside of the body cavity to move a proximal end of the first instrument away from a proximal end of the second instrument.

17. The method of claim 16, wherein the at least one instrument is bent prior to insertion through the at least one working channel.

18. The method of claim 16, further comprising using a tool to bend the at least one instrument in a controlled manner, the tool being keyed to a shape of the bendable region of the instrument.

19. The method of claim 16, further comprising longitudinally translating an actuation element disposed within the at least one instrument while the at least one instrument is bent.

20. The method of claim 16, wherein the at least one instrument includes a plurality of non-resilient bendable regions and further comprising forming multiple bends in the at least one instrument.

21. A method for manipulating an instrument, comprising:
inserting an elongate instrument through a working channel of an access device to position a distal end of the instrument within a body cavity; and
engaging a portion of the instrument disposed within the body cavity with a bending tool to bend the instrument at a non-resilient bendable region.

22. The method of claim 21, further comprising articulating the distal end of the instrument about an articulation joint after bending the instrument at the bendable region.
